(19) United States
(12) Patent Application Publication
(10) Pub. No.: US 2026/0330868 A1
(43) Pub. Date: Oct. 22, 2026

(54) DETECTION SYSTEM AND DETECTION DEVICE

(71) Applicant: Japan Display Inc., Tokyo (JP)

(72) Inventor: Naoshi GOTO, Tokyo (JP)

(21) Appl. No.: 19/559,005

(22) Filed: Mar. 6, 2026

Related U.S. Application Data
(63) Continuation of application No. PCT/JP2024/031166, filed on Aug. 30, 2024.

(30) Foreign Application Priority Data
Sep. 14, 2023 (JP) 2023-149613

(51) Int. Cl. G06V 40/13; G01L 1/14; G06V 40/12

(52) U.S. Cl. CPC G06V 40/1318; G01L 1/146; G06V 40/1306; G06V 40/1376

(57) ABSTRACT

According to an aspect, a detection system includes: a detection device having a contact region to be contacted by a predetermined part of a user; and a control device configured to operate to authenticate the user. The detection device includes: an optical sensor and a force sensor. The control device includes: an image processing circuit configured to generate a biometric information image based on the image of the predetermined part captured by the optical sensor; a storage circuit configured to store in advance the biometric information image and force information on force detected by the force sensor as a registered image and registered force information corresponding to the registered image; and a determination circuit configured to acquire an acquired image and acquired force information corresponding to the acquired image and determine whether the acquired image matches the registered image based on the acquired force information and the registered force information.

### CROSS-REFERENCE TO RELATED APPLICATION

This application claims the benefit of priority from Japanese Patent Application No. 2023-149613 filed on Sep. 14, 2023 and International Patent Application No. PCT/JP2024/031166 filed on Aug. 30, 2024, the entire contents of which are incorporated herein by reference.

### BACKGROUND

### 1. Technical Field

What is disclosed herein relates to a detection system and a detection device.

### 2. Description of the Related Art

Japanese Patent Application Laid-open Publication No. 2017-157421 (JP-A-2017-157421) discloses, as an example a detection system, a vein authentication system that authenticates a user, for example, by capturing an image of a vascular pattern of a finger of the user and comparing the vascular pattern with registered vascular patterns.

In the detection system of JP-A-2017-157421, the image of the finger or the like is captured using an imaging device configured with a complementary metal oxide semiconductor (CMOS) camera. In this case, the imaging device needs to be separated from the finger due to characteristics of the imaging device. Therefore, the system is difficult to be downsized.

For the foregoing reasons, there is a need for a downsizing of a detection system that authenticates users.

### SUMMARY

According to an aspect, a detection system includes: a detection device having a contact region to be contacted by a predetermined part of a user; and a control device configured to operate in an authentication mode to authenticate the user. The detection device includes: an optical sensor configured to capture an image of the predetermined part that includes a contact zone of the predetermined part in contact with the contact region; and a force sensor configured to detect force generated by contact of the predetermined part with the contact region. The control device includes: an image processing circuit configured to generate a biometric information image including biometric information of the user based on the image of the predetermined part captured by the optical sensor; a storage circuit configured to store in advance the biometric information image and force information on the force detected by the force sensor when the image of the predetermined part corresponding to the biometric information image is captured, as a registered image and registered force information corresponding to the registered image; and a determination circuit configured to acquire the biometric information image generated by the image processing circuit and the force information on the force detected by the force sensor when the image of the predetermined part corresponding to the biometric information image is captured, as an acquired image and acquired force information corresponding to the acquired image, and determine whether the acquired image matches the registered image based on the acquired force information and the registered force information, in the authentication mode.

According to an aspect, a detection device includes an optical sensor and a force sensor that are configured to be directly or indirectly contacted by a predetermined part of a user. The optical sensor has an imaging region to capture an image of the predetermined part. The force sensor has a detection region to detect force generated by being contacted by the predetermined part. The imaging region overlaps the detection region in plan view. Timing of outputting the image from the optical sensor is synchronized with timing of outputting the force from the force sensor.

### BRIEF DESCRIPTION OF THE DRAWINGS

FIG. 1 is a diagram illustrating a configuration of a detection system according to an embodiment of the present disclosure;

FIG. 2 is a side view of a detection device;

FIG. 3 is a plan view of a force sensor illustrated in FIG. 2;

FIG. 4 is a sectional view of the force sensor illustrated in FIG. 3;

FIG. 5 is a diagram illustrating a circuit configuration of the force sensor illustrated in FIG. 3;

FIG. 6 is a plan view of an optical sensor illustrated in FIG. 2;

FIG. 7 is a sectional view of the optical sensor illustrated in FIG. 6;

FIG. 8 is a diagram illustrating a circuit configuration of the optical sensor illustrated in FIG. 6;

FIG. 9 is a block diagram of the detection system;

FIG. 10 is a diagram illustrating force distribution information when a predetermined part is in contact with a contact region;

FIG. 11A is a diagram illustrating a vascular pattern of a first biometric information image;

FIG. 11B is a diagram illustrating the vascular pattern of a second biometric information image;

FIG. 11C is a diagram illustrating the vascular pattern of a third biometric information image;

FIG. 11D is a diagram illustrating the vascular pattern of a fourth biometric information image;

FIG. 11E is a diagram illustrating the vascular pattern of a fifth biometric information image;

FIG. 12 is a flowchart of a process executed by a control device in a registration mode;

FIG. 13 is a flowchart of a process executed by the control device in an authentication mode;

FIG. 14 is a side view of the detection device according to a modification of the embodiment of the present disclosure;

FIG. 15 is a schematic diagram illustrating a configuration of a force sensor according to the modification of the present disclosure; and

FIG. 16 is a sectional view of the force sensor illustrated in FIG. 15.

### DETAILED DESCRIPTION

The following describes an embodiment of the present disclosure with reference to the drawings. The present disclosure is not limited to the description of the embodiment given below. Components described below include those that are easily conceivable by those skilled in the art or those that are substantially identical thereto. In addition, the components described below can be combined as appropriate.

What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same element as that described with reference to an already mentioned drawing is denoted by the same reference numeral through the present specification and the drawings, and detailed description thereof may not be repeated where appropriate.

An X direction and a Y direction illustrated in the drawings correspond to directions parallel to a plate surface of a substrate included in a detection device 10. A +X side and a -X side in the X direction and a +Y side and a -Y side in the Y direction correspond to lateral sides of the detection device 10. A Z direction corresponds to a thickness direction of the detection device 10. A +Z side in the Z direction corresponds to a front side of the detection device 10, and a -Z side in the Z direction corresponds to a back side of the detection device 10. In this specification, "plan view" refers to viewing the detection device 10 from the +Z side toward the -Z side along the Z direction. The X, Y, and Z directions are examples, and the present disclosure is not limited to these directions.

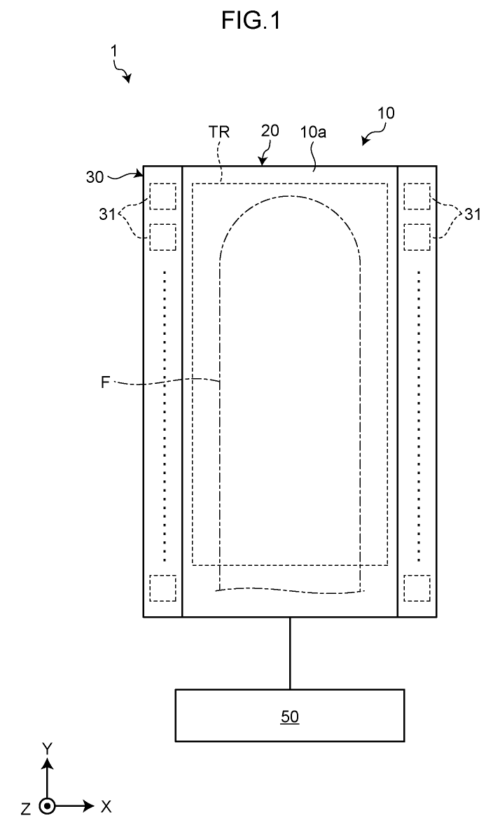

FIG. 1 is a diagram illustrating a configuration of a detection system 1 according to the embodiment of the present disclosure. The detection system 1 is a system that detects biometric information of a user and authenticates the identity of the user. The biometric information is a vascular pattern. The biometric information may be a fingerprint pattern. The detection system 1 includes the detection device 10 and a control device 50. The detection device 10 and the control device 50 are electrically coupled to each other.

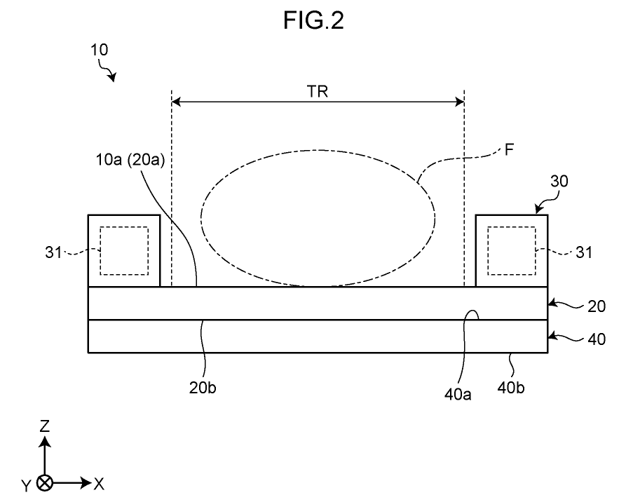

FIG. 2 is a side view of the detection device 10. As illustrated in FIGS. 1 and 2, the detection device 10 has a contact region TR on the front surface thereof (hereinafter, referred to as a "contact surface 10a") to be contacted by a predetermined part F of a user. The predetermined part F is a finger of the user. The predetermined part F contacts the contact region TR while extending along the Y direction. The predetermined part F may be the entire hand or a part of a hand (such as a palm) of the user.

The detection device 10 includes a force sensor 20, a light source device 30, and an optical sensor 40. The optical sensor 40 and the force sensor 20 are each a flat plate and rectangular in shape with the Y direction as the longitudinal direction in plan view. The optical sensor 40 and the force sensor 20 are arranged in the Z direction to overlap each other.

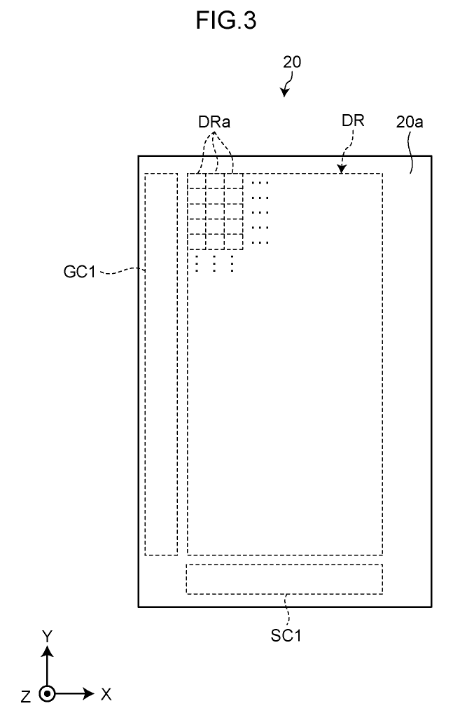

FIG. 3 is a plan view of the force sensor 20 illustrated in FIG. 2. The force sensor 20 detects force generated by contact of the predetermined part F with the contact region TR. The force sensor 20 has a detection region DR for detecting the force on the front surface thereof (hereinafter, referred to as a "detection surface 20a"). The detection surface 20a of the force sensor 20 corresponds to the contact surface 10a of the detection device 10. The detection region DR matches the contact region TR. The predetermined part F of a user comes into direct contact with the detection region DR of the force sensor 20.

The detection region DR is divided into a plurality of individual detection regions DRa. The individual detection regions DRa are arranged in a matrix having a row-column configuration along the X and Y directions. The force sensor 20 includes a first gate line drive circuit GC1 and a first signal line selection circuit SC1 provided at positions outside the detection region DR in plan view.

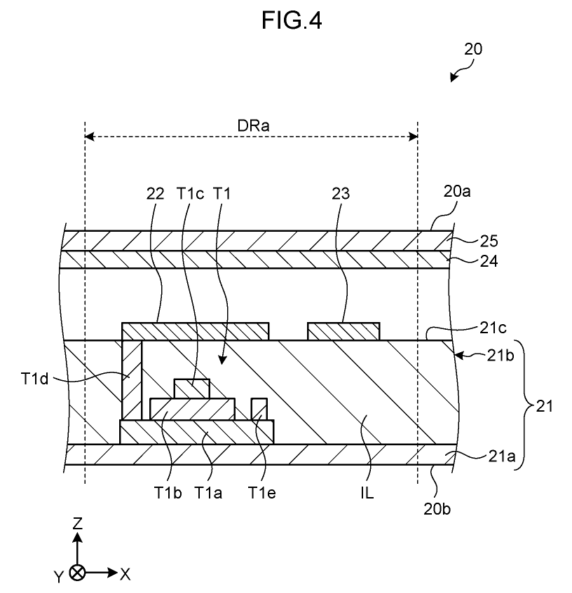

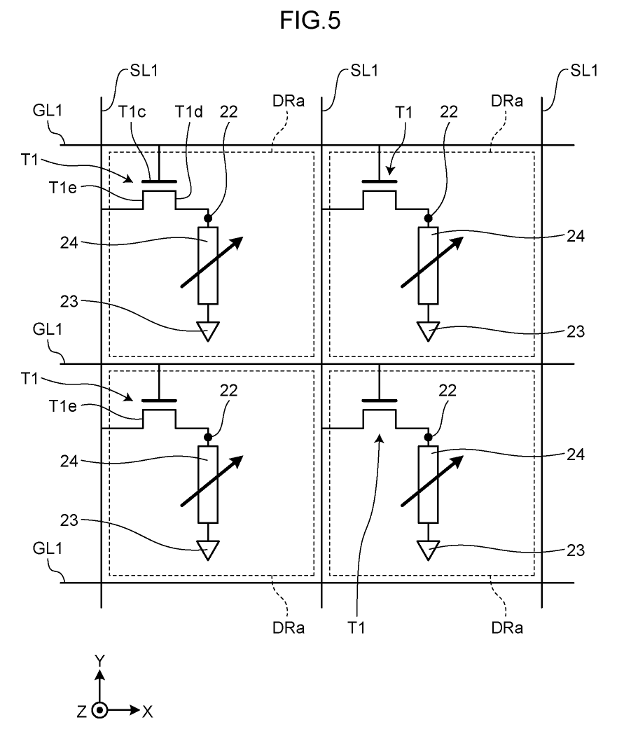

FIG. 4 is a sectional view of the force sensor 20 illustrated in FIG. 3. FIG. 5 is a diagram illustrating a circuit configuration of the force sensor 20 illustrated in FIG. 3.

As illustrated in FIG. 4, the force sensor 20 includes an array substrate 21, an array electrode 22, a common electrode 23, a first sensor layer 24, and a first protective layer 25. The first sensor layer 24 corresponds to a "sensor layer".

The array substrate 21 includes a first substrate 21a and an array layer 21b. The first substrate 21a has an electrically insulating property. The first substrate 21a has relatively high rigidity. The first substrate 21a is, for example, a glass substrate, a resin substrate, or a resin film. The back surface of the first substrate 21a corresponds to a back surface 20b of the force sensor 20.

The array layer 21b is positioned on the front surface of the first substrate 21a. The array layer 21b includes an insulating layer IL illustrated in FIG. 4, a first transistor T1 illustrated in FIGS. 4 and 5, a first gate line GL1 and a first signal line SL1 illustrated in FIG. 5, and the first gate line drive circuit GC1 and the first signal line selection circuit SC1 illustrated in FIG. 3. The first gate line GL1, the first signal line SL1, the first transistor T1, the first gate line drive circuit GC1, and the first signal line selection circuit SC1 are arranged on the front surface of the first substrate 21a.

The insulating layer IL illustrated in FIG. 4 has an electrically insulating property and covers the first gate line GL1, the first signal line SL1, the first transistor T1, the first gate line drive circuit GC1, and the first signal line selection circuit SC1. The front surface of the insulating layer IL has a planar shape. The front surface of the insulating layer IL corresponds to a front surface 21c of the array substrate 21. The front surface 21c of the array substrate 21 corresponds to a "first surface".

As illustrated in FIG. 5, a plurality of the first gate lines GL1 are disposed on the front surface of the first substrate 21a while extending along the X direction and being arranged along the Y direction. The first gate lines GL1 are electrically coupled to the first gate line drive circuit GC1. A plurality of the first signal lines SL1 are disposed on the front surface of the first substrate 21a while extending along the Y direction and being arranged along the X direction. The first signal lines SL1 are electrically coupled to the first signal line selection circuit SC1. The first signal line selection circuit SC1 is a multiplexer, for example. A region surrounded by adjacent two of the first gate lines GL1 and adjacent two of the first signal lines SL1 corresponds to each of the individual detection regions DRa.

One array electrode 22 and one common electrode 23 are arranged in each of the individual detection regions DRa. That is, the array electrodes 22 are arranged in a matrix having a row-column configuration in the detection region DR in plan view, and the common electrodes 23 are arranged in a matrix having a row-column configuration in the detection region DR in plan view.

One first transistor T1 is disposed in each of the individual detection regions DRa. As illustrated in FIG. 4, the first transistor T1 includes a semiconductor layer T1a, a gate insulating film T1b, a gate electrode T1c, a drain electrode T1d, and a source electrode T1e.

As illustrated in FIG. 5, the gate electrode T1c is electrically coupled to the first gate line GL1. The drain electrode T1d is electrically coupled to the array electrode 22. The source electrode T1e is electrically coupled to the first signal line SL1.

As illustrated in FIG. 4, the array electrode 22 and the common electrode 23 are arranged on the front surface 21c of the array substrate 21. The array electrode 22 and the common electrode 23 are formed of a metal material such as indium tin oxide (ITO). The array electrode 22 and the common electrode 23 may be formed of metal materials different from each other.

The first sensor layer 24 faces the front surface 21c of the array substrate 21 and is disposed at a distance toward the +Z side from the array substrate 21. The first sensor layer 24 is supported by a spacer (not illustrated) disposed on the array substrate 21. The first sensor layer 24 faces the array electrode 22 and the common electrode 23 while being separated therefrom. The first sensor layer 24 has flexibility to be deformed by the contact of the predetermined part F with the detection region DR.

The first sensor layer 24 contains a plurality of conductive fine particles and is formed of a resin material having a relatively high electrically insulating property. The fine particles are dispersed in the first sensor layer 24 and are separated from one another. The first sensor layer 24 is deformed by the contact of the predetermined part F of a user with the detection region DR (contact region TR), and is compressed when the first sensor layer 24 contacts the array substrate 21.

The compression of the first sensor layer 24 reduces the spacing between the fine particles and reduces the electrical resistance value of the first sensor layer 24. The electrical resistance value of the first sensor layer 24 decreases as the amount of compression of the first sensor layer 24 increases. Thus, the electrical resistance value of the first sensor layer 24 is changed by the contact of the predetermined part F with the detection region DR (contact region TR).

The first protective layer 25 is disposed on the front surface of the first sensor layer 24. The first protective layer 25 has an electrically insulating property. The first protective layer 25 has flexibility to be deformed together with the first sensor layer 24 by the contact of the predetermined part F with the detection region DR. The front surface of the first protective layer 25 corresponds to the detection surface 20a of the force sensor 20.

The force sensor 20 has a light-transmitting property. Specifically, among the components of the force sensor 20, at least components that overlap the detection region DR in plan view are made of a light-transmitting material.

The following describes operations of the force sensor 20.

A constant amount of current is supplied to the common electrode 23 from the control device 50 via a conducting wire (not illustrated) disposed on the array substrate 21. The first gate line drive circuit GC1 sequentially or simultaneously selects the first gate lines GL1 based on a control signal output from the control device 50, and supplies a gate drive signal that drives the gate of the first transistor T1 to each of the selected first gate lines GL1. The first signal line selection circuit SC1 selects the first signal line SL1 based on a control signal output from the control device 50, and electrically couples the selected first signal line SL1 to the control device 50. As a result, the control device 50 is supplied with the current through the array electrode 22 in the individual detection region DRa corresponding to the first gate line GL1 supplied with the gate drive signal and the selected first signal line SL1.

When the predetermined part F of a user is not in contact with the individual detection region DRa, the first sensor layer 24 is not deformed, and the common electrode 23 is not electrically coupled to the array electrode 22. In this case, no current flows through the array electrode 22, and the current value of the array electrode 22 is zero.

In contrast, when the predetermined part F of a user contacts the individual detection region DRa, and the first sensor layer 24 is deformed and contacts the array substrate 21, the common electrode 23 is electrically coupled to the array electrode 22 via the first sensor layer 24. In this case, a current is supplied from the common electrode 23 to the array electrode 22 via the first sensor layer 24. The amount of compression of the first sensor layer 24 increases and the electrical resistance value of the first sensor layer 24 decreases as the force acting on the individual detection region DRa increases. Therefore, the current value of the array electrode 22 increases as the force acting on the individual detection region DRa increases.

Thus, the current value of the array electrode 22 changes depending on the magnitude of the force acting on the individual detection region DRa. The current flowing through the array electrode 22 is supplied to the control device 50. The control device 50 acquires the current value of the array electrode 22 as the magnitude of the force (force value) acting on the individual detection region DRa. That is, the force sensor 20 detects the magnitude of the force acting on the individual detection region DRa, and outputs it to the control device 50. As described above, the individual detection regions DRa are arranged in a matrix having a row-column configuration in the detection region DR. Therefore, the force sensor 20 supplies the current to the control device 50 through the array electrode 22 for each of the individual detection regions DRa. As a result, the control device 50 acquires the output of the force sensor 20 as force distribution information of the detection region DR. That is, the force sensor 20 detects the force acting on the detection region DR, and outputs it as the force distribution information to the control device 50.

As illustrated in FIGS. 1 and 2, the light source device 30 is disposed on the contact surface 10a side. The light source device 30 includes a plurality of light sources 31. The light sources 31 are light-emitting diodes (LEDs), for example. The light sources 31 are arranged along the Y direction on both sides of the contact region TR (detection region DR) in the X direction.

The light sources 31 emit light toward the predetermined part F in contact with the contact region TR. The light of the light sources 31 is reflected in the predetermined part F and enters the force sensor 20 as reflected light. As described above, the force sensor 20 has a light-transmitting property. Therefore, the reflected light passes through the force sensor 20 and enters the optical sensor 40.

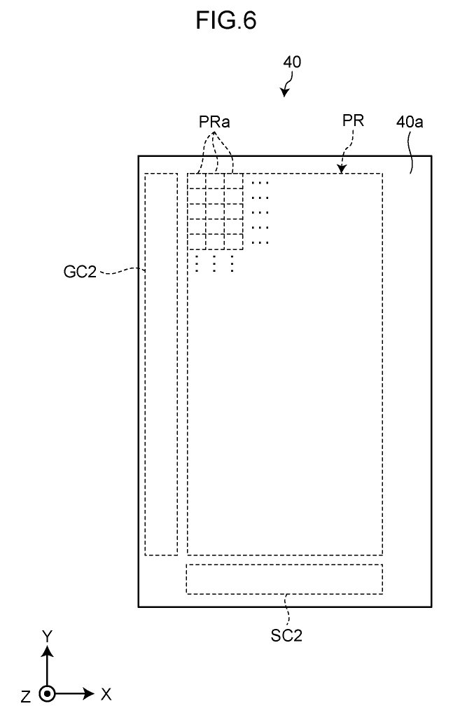

FIG. 6 is a plan view of the optical sensor 40 illustrated in FIG. 2. The optical sensor 40 captures an image of the predetermined part F in contact with the contact region TR. The image of the predetermined part F captured by the optical sensor 40 includes a contact zone of the predetermined part F in contact with the contact region TR. As illustrated in FIG. 2, the optical sensor 40 is positioned on an opposite surface (that is, the back surface 20b) of the force sensor 20 opposite the detection surface 20a. That is, the predetermined part F of a user indirectly contacts the optical sensor 40 with the force sensor 20 interposed therebetween.

As illustrated in FIG. 6, the optical sensor 40 has an imaging region PR for capturing the image of the predetermined part F on the front surface (hereinafter, referred to as an "imaging surface 40a"). The imaging region PR matches the detection region DR of the force sensor 20 in plan view.

The imaging region PR is divided into a plurality of individual imaging regions PRa. The individual imaging regions PRa are arranged in a matrix having a row-column configuration along the X and Y directions. The optical sensor 40 includes a second gate line drive circuit GC2 and a second signal line selection circuit SC2 at positions outside the imaging region PR in plan view.

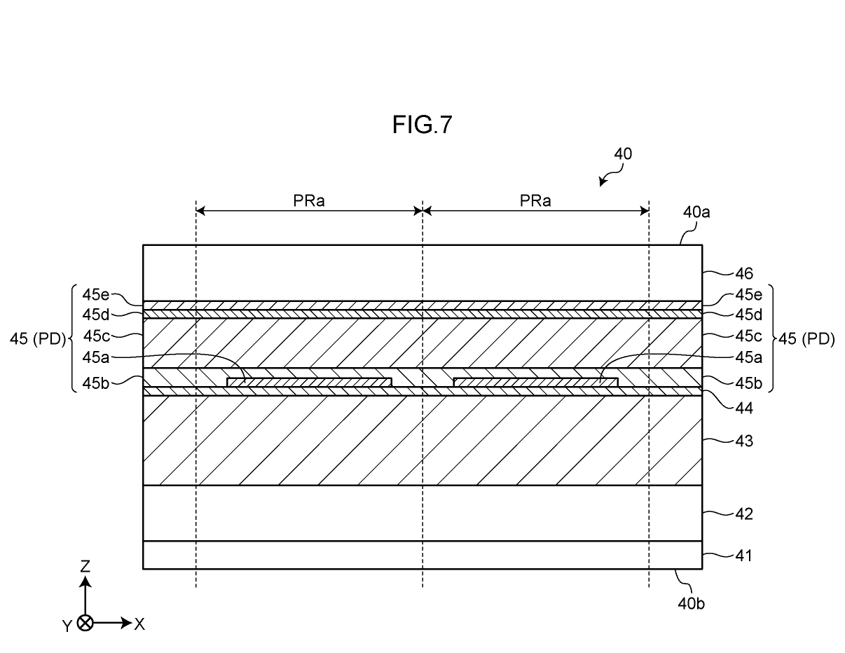

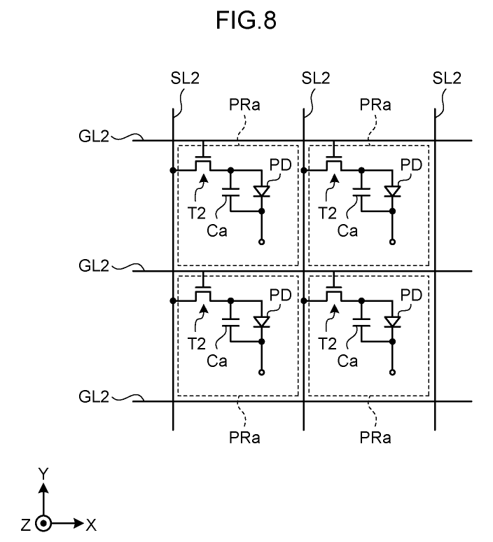

FIG. 7 is a sectional view of the optical sensor 40 illustrated in FIG. 6. FIG. 8 is a diagram illustrating a circuit configuration of the optical sensor 40 illustrated in FIG. 6.

As illustrated in FIG. 7, the optical sensor 40 includes a second substrate 41, a circuit forming layer 42, an organic insulating film 43, an inorganic insulating film 44, a second sensor layer 45, and a second protective layer 46.

The second substrate 41 has an electrically insulating property. The second substrate 41 has relatively high rigidity. The second substrate 41 is, for example, a glass substrate, a resin substrate, or a resin film. The back surface of the second substrate 41 corresponds to a back surface 40b of the optical sensor 40.

The circuit forming layer 42 is disposed on the front surface of the second substrate 41. The second gate line drive circuit GC2 and the second signal line selection circuit SC2 illustrated in FIG. 6, and a second gate line GL2, a second signal line SL2, and a second transistor T2 illustrated in FIG. 8 are arranged in the circuit forming layer 42.

The organic insulating film 43 is disposed on the front surface of the circuit forming layer 42. The organic insulating film 43 is formed of an organic insulating material having an electrically insulating property. The organic insulating film 43 covers the second gate line GL2, the second signal line SL2, the second transistor T2, the second gate line drive circuit GC2, and the second signal line selection circuit SC2.

The inorganic insulating film 44 is disposed on the front surface of the organic insulating film 43. The inorganic insulating film 44 is formed of an inorganic insulating material having an electrically insulating property.

As illustrated in FIG. 8, a plurality of the second gate lines GL2 are disposed on the front surface of the second substrate 41 while extending along the X direction and being arranged along the Y direction. The second gate lines GL2 are electrically coupled to the second gate line drive circuit GC2. A plurality of the second signal lines SL2 are disposed on the front surface of the second substrate 41 while extending along the Y direction and being arranged along the X direction. The second signal lines SL2 are electrically coupled to the second signal line selection circuit SC2. The second signal line selection circuit SC2 is a multiplexer, for example. A region surrounded by adjacent two of the second gate lines GL2 and adjacent two of the second signal lines SL2 corresponds to each of the individual imaging regions PRa.

Each of the individual imaging regions PRa includes a capacitive element Ca, the second transistor T2, and a photodiode PD. That is, a plurality of the photodiodes PD overlap the contact region TR (detection region DR) and are arranged in a matrix having a row-column configuration in plan view.

The capacitive element Ca is capacitance (sensor capacitance) generated in the photodiode PD and is equivalently electrically coupled in parallel to the photodiode PD.

The gate of the second transistor T2 is electrically coupled to the second gate line GL2. One of the source and the drain of the second transistor T2 is electrically coupled to the second signal line SL2. The other of the source and the drain of the second transistor T2 is electrically coupled to the anode of the photodiode PD and the capacitive element Ca.

The second sensor layer 45 illustrated in FIG. 7 includes a lower electrode 45a, a lower buffer layer 45b, an active layer 45c, an upper buffer layer 45d, and an upper electrode 45e. A portion of the second sensor layer 45 overlapping the lower electrode 45a in plan view corresponds to the photodiode PD illustrated in FIG. 8.

A plurality of the lower electrodes 45a are arranged on the front surface of the inorganic insulating film 44. One lower electrode 45a is disposed in each of the individual imaging regions PRa. That is, a plurality of the lower electrodes 45a are arranged in a matrix having a row-column configuration in the imaging region PR in plan view. The lower electrode 45a corresponds to an anode electrode of the photodiode PD. The lower electrode 45a is formed of a metal material such as indium tin oxide (ITO).

The lower buffer layer 45b is disposed on the front surface of the inorganic insulating film 44 while covering the lower electrode 45a. The lower buffer layer 45b serves as a hole transport layer. The lower buffer layer 45b is formed, for example, of tungsten oxide (WO₃) or molybdenum oxide.

The active layer 45c is disposed on the front surface of the lower buffer layer 45b. The active layer 45c changes in electrical property (such as electrical resistance value) depending on light incident thereon. An organic semiconductor is used as the active layer 45c. That is, the photodiode PD is an organic photodiode (OPD). The active layer 45c has a bulk heterostructure containing, for example, a mixture of a p-type organic semiconductor and an n-type fullerene derivative ((6,6)-phenyl-C₆₁-butyric acid methyl ester (PCBM)) that is an n-type organic semiconductor.

The upper buffer layer 45d is disposed on the front surface of the active layer 45c. The upper buffer layer 45d serves as an electron transport layer. The upper buffer layer 45d is formed, for example, of ethoxylated polyethyleneimine (PEIE).

The upper electrode 45e is disposed on the front surface the upper buffer layer 45d. The upper electrode 45e is a cathode electrode of the photodiode PD. The upper electrode 45e is continuously provided over the entire imaging region PR in plan view. The upper electrode 45e faces the lower electrodes 45a with the upper buffer layer 45d, the active layer 45c, and the lower buffer layer 45b interposed therebetween. The upper electrode 45e is formed of a metal material such as indium tin oxide (ITO).

The configuration of the photodiode PD illustrated in FIGS. 7 and 8 is an example. The configuration of the photodiode PD is not limited to that described above. For example, the upper electrode 45e may correspond to the anode electrode, and the lower electrode 45a may correspond to the cathode electrode.

The second protective layer 46 is disposed on the front surface of the second sensor layer 45. The second protective layer 46 has an electrically insulating property. The front surface of the second protective layer 46 corresponds to the imaging surface 40a of the optical sensor 40.

The following describes operations of the optical sensor 40.

A predetermined voltage is applied to the anode (lower electrode 45a) of the photodiode PD from the control device 50 via a conductor (not illustrated) positioned in the circuit forming layer 42. The second gate line drive circuit GC2 sequentially or simultaneously selects the second gate lines GL2 based on a control signal output from the control device 50, and supplies the gate drive signal that drives the gate of the second transistor T2 to each of the selected second gate lines GL2. The second signal line selection circuit SC2 selects the second signal line SL2 based on a control signal output from the control device 50, and electrically couples the selected second signal line SL2 to the control device 50. As a result, the control device 50 is supplied with a current corresponding to capacitance stored in the capacitive element Ca in the individual imaging region PRa corresponding to the second gate line GL2 supplied with the gate drive signal and the selected second signal line SL2.

When the predetermined part F of a user is in contact with the contact region TR, the light of the light sources 31 reflected by the predetermined part F (reflected light) enters the individual imaging region PRa. The amount of the reflected light varies depending on the shape and the internal structure (such as positions of blood vessels) of the predetermined part F. The capacitance of the capacitive element Ca changes with the amount of the reflected light.

The current corresponding to the capacitance of the capacitive element Ca is supplied to the control device 50 as described above. In addition, the individual imaging regions PRa are arranged in a matrix having a row-column configuration in the imaging region PR, as described above. That is, the optical sensor 40 supplies, to the control device 50, the current corresponding to the capacitance of the capacitive element Ca for each of the individual imaging regions PRa. As a result, when the predetermined part F of a user is in contact with the contact region TR, the control device 50 acquires currents corresponding to capacitance of a plurality of the capacitive elements Ca as the image of the predetermined part F in contact with the contact region TR. That is, when the predetermined part F of the user is in contact with the contact region TR, the optical sensor 40 outputs the image of the predetermined part F to the control device 50.

The timing of outputting the force distribution information from the force sensor 20 is synchronized with the timing of outputting the image of the predetermined part F from the optical sensor 40. Specifically, the interval at which the force sensor 20 outputs the force distribution information is equal to the interval at which the optical sensor 40 outputs the image of the predetermined part F. The time when the force sensor 20 outputs the force distribution information may be the same as the time when the optical sensor 40 outputs the image of the predetermined part F. This timing allows the control device 50 to associate the force distribution information output by the force sensor 20 with the image of the predetermined part F output by the optical sensor 40. The force sensor 20 may continuously output the force distribution information, and the optical sensor 40 may continuously output the images of the predetermined part F.

As described above, the optical sensor 40 captures the image of the predetermined part F including the contact zone of the predetermined part F in contact with the contact region TR. Therefore, compared with a case where the detection device 10 is provided with a camera for imaging the predetermined part F and the camera images the predetermined part F while the camera is positioned at a distance from the predetermined part F, the detection device 10 need not be provided with a support stand for supporting the predetermined part F. Thus, the detection device 10 can be downsized.

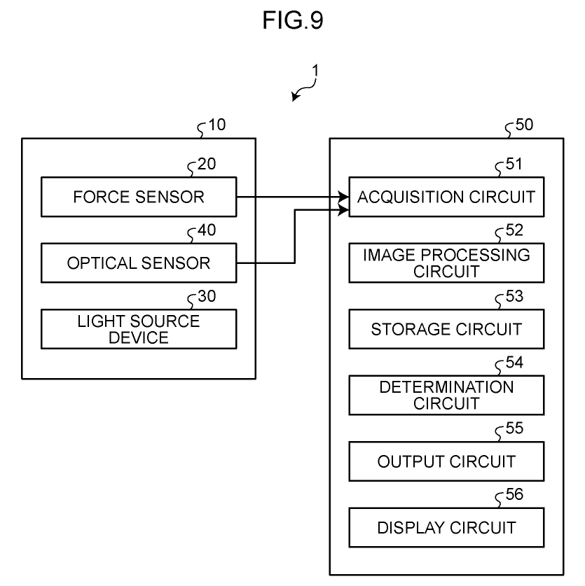

FIG. 9 is a block diagram of the detection system 1. The control device 50 is a computer, and includes, for example, a central processing unit (CPU), a read-only memory (ROM), a random-access memory (RAM), a storage circuit 53 to be described later, an input interface, and an output interface. The CPU, the ROM, the RAM, and the storage circuit 53 are coupled via an internal bus. The ROM stores therein computer programs, such as the Basic Input/Output System (BIOS). The storage circuit 53 is, for example, a hard disk drive (HDD) or a flash memory, and stores therein an operating system program and application programs. The CPU performs various functions by executing computer programs stored in the ROM or the storage circuit 53 while using the RAM as a work area.

The control device 50 includes an acquisition circuit 51, an image processing circuit 52, the storage circuit 53, a determination circuit 54, an output circuit 55, and a display circuit 56.

The acquisition circuit 51 acquires the force distribution information output from the force sensor 20 and the image of the predetermined part F output from the optical sensor 40.

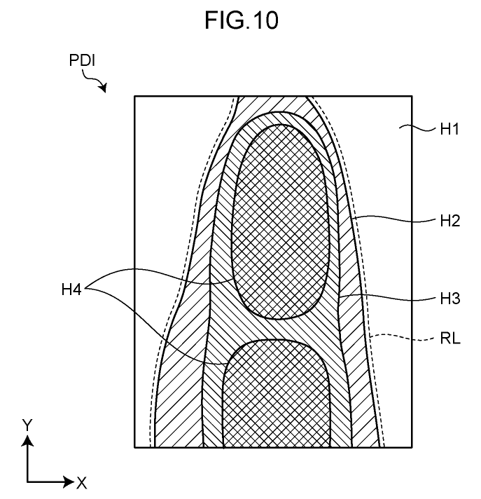

FIG. 10 is a diagram illustrating force distribution information PDI when the predetermined part F is in contact with the contact region TR. The force distribution information PDI is divided into four force zones. The magnitude of force in the force distribution information PDI is larger in the order of a first force zone H1, a second force zone H2, a third force zone H3, and a fourth force zone H4. The first force zone H1 has a force value of zero. The force distribution information PDI illustrated in FIG. 10 is an example. The number and the shapes of the force zones are not limited to those in the force distribution information PDI illustrated in FIG. 10.

The acquisition circuit 51 illustrated in FIG. 9 acquires the image of the predetermined part F captured by the optical sensor 40 in a synchronized state with the force distribution information output by the force sensor 20.

The image processing circuit 52 generates a biometric information image including the biometric information of a user based on the image of the predetermined part F captured by the optical sensor 40. The biometric information is the vascular pattern (vein pattern).

The image processing circuit 52 acquires the image of the predetermined part F captured by the optical sensor 40 from the acquisition circuit 51. The image processing circuit 52 extracts the vascular pattern (vein pattern) of the predetermined part F by adjusting the brightness, contrast, and the like of the image of the predetermined part F, and generates the biometric information image.

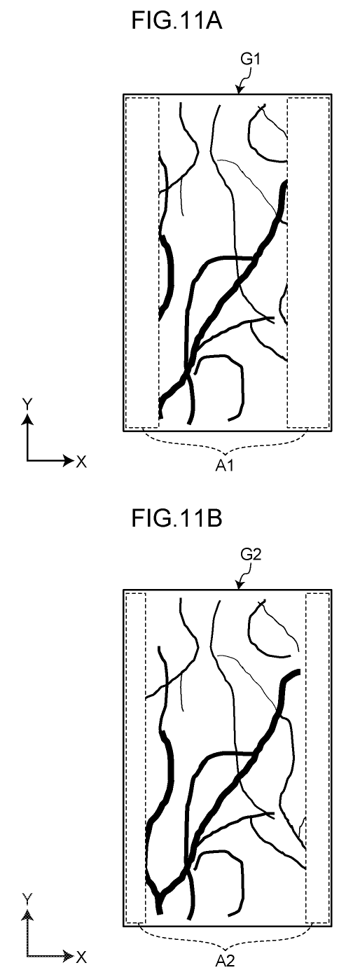

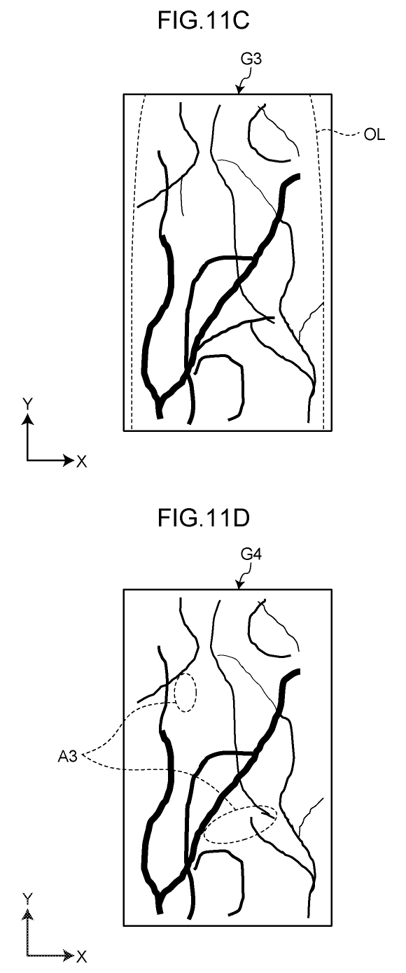

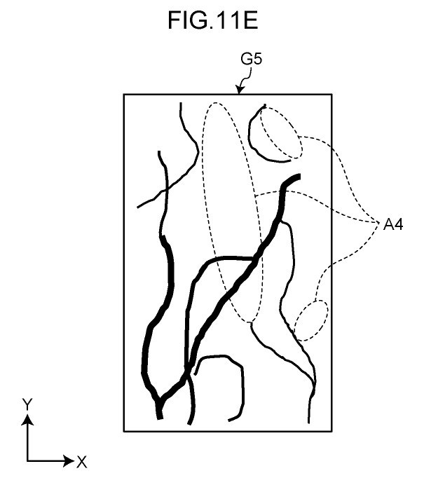

FIG. 11A is a diagram illustrating the vascular pattern of a first biometric information image G1. FIG. 11B is a diagram illustrating the vascular pattern of a second biometric information image G2. FIG. 11C is a diagram illustrating the vascular pattern of a third biometric information image G3. FIG. 11D is a diagram illustrating the vascular pattern of a fourth biometric information image G4. FIG. 11E is a diagram illustrating the vascular pattern of a fifth biometric information image G5.

The maximum value of force included in the force distribution information output by the force sensor 20, which is synchronized with the original image of the predetermined part F, becomes larger in the order of the first biometric information image G1, the second biometric information image G2, the third biometric information image G3, the fourth biometric information image G4, and the fifth biometric information image G5. Of the first biometric information image G1, the second biometric information image G2, the third biometric information image G3, the fourth biometric information image G4, and the fifth biometric information image G5, the first biometric information image G1 has the smallest maximum force value, and the fifth biometric information image G5 has the largest maximum force value. That is, the pressing force with which the predetermined part F is pressed against the contact region TR when the original image of the predetermined part F is captured becomes larger in the order of the first biometric information image G1, the second biometric information image G2, the third biometric information image G3, the fourth biometric information image G4, and the fifth biometric information image G5.

Consequently, in the first biometric information image G1 illustrated in FIG. 11A, the vascular pattern disappears or becomes blurred on both sides in the X direction as compared with the third biometric information image G3 illustrated in FIG. 11C. In regions A1 located on both sides in the X direction of the first biometric information image G1 illustrated in FIG. 11A, the vascular pattern disappears as compared with the third biometric information image G3 illustrated in FIG. 11C.

In the second biometric information image G2 illustrated in FIG. 11B, the vascular pattern disappears or becomes blurred on both sides in the X direction as compared with the third biometric information image G3 illustrated in FIG. 11C. In regions A2 located on both sides in the X direction of the second biometric information image G2 illustrated in FIG. 11B, the vascular pattern disappears as compared with the third biometric information image G3 illustrated in FIG. 11C. The length in the X direction of the regions A2 is shorter than the length in the X direction of the regions A1 in FIG. 11A.

Furthermore, in the fourth biometric information image G4 illustrated in FIG. 11D, the vascular pattern disappears in regions A3 illustrated in FIG. 11D as compared with the third biometric information image G3 illustrated in FIG. 11C. This result is caused by deterioration of the blood flow condition at the predetermined part F because the pressing force corresponding to the fourth biometric information image G4 illustrated in FIG. 11D is greater than the pressing force corresponding to the third biometric information image G3 illustrated in FIG. 11C.

In the fifth biometric information image G5 illustrated in FIG. 11E, the vascular pattern disappears in regions A4 illustrated in FIG. 11E as compared with the third biometric information image G3 illustrated in FIG. 11D. This result is caused by the deterioration of the blood flow condition at the predetermined part F because the pressing force corresponding to the fifth biometric information image G5 illustrated in FIG. 11E is greater than the pressing force corresponding to the fourth biometric information image G4 illustrated in FIG. 11D.

Thus, the vascular pattern changes in accordance with the pressing force corresponding to the biometric information image. As described above, the greater the pressing force corresponding to the biometric information image, the greater the force output by the force sensor 20. That is, the vascular pattern changes depending on the force output by the force sensor 20.

The image processing circuit 52 may extract the contour of the predetermined part F in the biometric information image. Specifically, the image processing circuit 52 first extracts the contour of the predetermined part F in the force distribution information synchronized with the original image of the predetermined part F in the biometric information image. For example, a boundary RL between the first force zone H1 where the force value is zero and the second force zone H2 in the force distribution information PDI illustrated in FIG. 10 is extracted as the contour of the predetermined part F. Furthermore, the image processing circuit 52 extracts, as the contour of the predetermined part F in the biometric information image, a line (for example, a dashed line OL illustrated in FIG. 11C) corresponding to the contour (boundary RL) of the predetermined part F in the force distribution information from the biometric information image generated from the image of the predetermined part F synchronized with the force distribution information from which the contour of the predetermined part F has been extracted.

By extracting the contour of the predetermined part F as described above, the image processing circuit 52 can clearly distinguish between the contour of the predetermined part F and the vascular pattern in the biometric information image. The vascular pattern is located inside the contour of the predetermined part F. Thus, the image processing circuit 52 can appropriately extract the vascular pattern.

The storage circuit 53 stores therein the biometric information image and the force distribution information that has been output by the force sensor 20 in association with each other (details will be described later).

The determination circuit 54 uses the biometric information image to determine whether a user is the person himself/herself (details will be described later). If the determination circuit 54 determines that the user is the person himself/herself, the user's identity is authenticated.

The output circuit 55 outputs the determination result of the determination circuit 54. The output circuit 55 outputs the determination result to the display circuit 56. The display circuit 56 displays the determination result. The display circuit 56 is, for example, a display.

The control device 50 operates in one of a registration mode and an authentication mode. The registration mode is a mode in which a user registers himself/herself with the detection system 1 so that the user can use the detection system 1. The authentication mode is a mode in which the detection system 1 authenticates the user's identity.

Switching between the registration mode and the authentication mode is performed, for example, by a user entering a selection instruction on an input device (such as a keyboard or a touch panel (not illustrated)) provided on the control device 50.

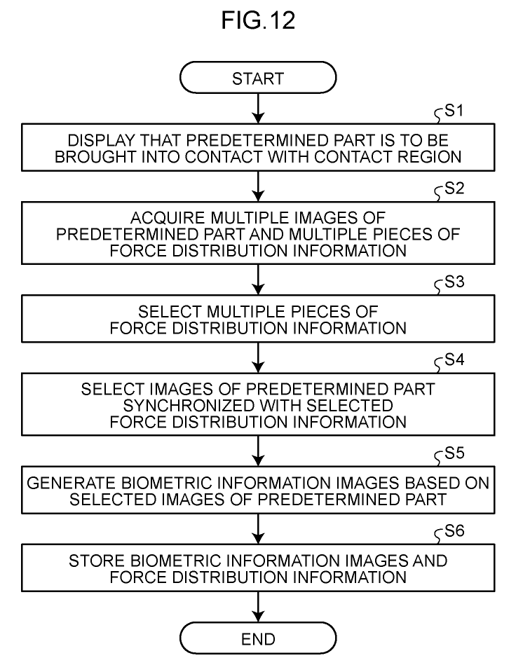

FIG. 12 is a flowchart of a process executed by the control device 50 in the registration mode. The control device 50 executes the process of the flowchart illustrated in FIG. 12 in response to the selection of the registration mode.

At Step S1, the control device 50 (display circuit 56) displays that the predetermined part F is to be brought into contact with the contact region TR. At this time, the control device 50 also displays on the display circuit 56 that the pressing force of the predetermined part F against the contact region TR is to be changed.

The user brings the predetermined part F into contact with the contact region TR and changes the pressing force in accordance with the display of the display circuit 56. At this time, the optical sensor 40 captures a plurality of images of the predetermined part F and outputs them to the control device 50, as described above. The force sensor 20 detects a plurality of values of the force acting on the detection region DR and outputs them to the control device 50. As described above, the images of the predetermined part F output from the optical sensor 40 are synchronized with the values of the force output from the force sensor 20.

Then, at Step S2, the control device 50 (acquisition circuit 51) acquires a plurality of images of the predetermined part F output by the optical sensor 40 and a plurality of pieces of the force distribution information output by the force sensor 20. By the user changing the pressing force of the predetermined part F against the contact region TR as described above, the multiple images of the predetermined part F acquired by the control device 50 differ from one another, and the multiple pieces of the force distribution information acquired by the control device 50 differ from one another.

Furthermore, at Step S3, the control device 50 selects a plurality of pieces of the force distribution information from the acquired multiple pieces of the force distribution information. First, the control device 50 (image processing circuit 52) calculates the area in the detection region DR in which the force falls within a predetermined force range, for each of the multiple pieces of the force distribution information acquired at Step S2. The predetermined force range is a range of force values in which the blood flow condition at the predetermined part F in contact with the contact region TR is appropriate and the image processing circuit 52 can appropriately extract the vascular pattern from the image of the predetermined part F. For example, in the force distribution information PDI illustrated in FIG. 10, if the first force zone H1 and the second force zone H2 are outside the predetermined force range, and the third force zone H3 and the fourth force zone H4 are within the predetermined force range, the control device 50 calculates the area of a zone obtained by summing the third force zone H3 and the fourth force zone H4 in the detection region DR.

The control device 50 selects, from the multiple pieces of the force distribution information acquired at Step S2, a predetermined number (an integer equal to or greater than 2, such as 5) of pieces of the force distribution information in each of which the calculated area falls within a predetermined area range. The predetermined area range is an area range within which the determination circuit 54 can appropriately perform determination to be described later. If the number of pieces of the force distribution information in each of which the calculated area is within the predetermined area range exceeds the predetermined number, the control device 50 may select the force distribution information in descending order of the calculated area. If the number of pieces of the force distribution information in each of which the calculated area is within the predetermined area range is one, or if the number of pieces of the force distribution information selected does not reach the predetermined number, the control device 50 may return to Step S1.

Furthermore, at Step S4, the control device 50 selects images of the predetermined part F that are synchronized with the selected force distribution information from among the multiple images of the predetermined part F acquired at Step S2. The number of the selected images of the predetermined part F is equal to the number (predetermined number) of the selected pieces of the force distribution information.

Then, at Step S5, the control device 50 (image processing circuit 52) generates the biometric information images based on the images of the predetermined part F selected at Step S4. The biometric information images generated by the control device 50 at Step S4 correspond to the biometric information images illustrated in FIGS. 11A, 11B, 11C, 11D and 11E, for example. The biometric information images correspond to the pieces of the force distribution information synchronized with the original images of the predetermined part F.

Furthermore, at Step S6, the control device 50 (storage circuit 53) stores the biometric information images and the force distribution information. Specifically, the control device 50 associates the biometric information images generated at Step S5 with the multiple pieces of the force distribution information (the force distribution information selected at Step S3) corresponding to the biometric information images, and stores the biometric information images and the multiple pieces of the force distribution information.

The control device 50 also stores the multiple biometric information images and the multiple pieces of the force distribution information in association with identification information (such as an identification number) that identifies the user. As a result, the control device 50 can identify the user corresponding to the multiple biometric information images and the multiple pieces of the force distribution information stored in the storage circuit 53 as the user corresponding to the identification information. The control device 50 (acquisition circuit 51) acquires the identification information when the user enters the identification information into the input device. When the process at Step S6 ends, the control device 50 ends the computer program.

Thus, in the registration mode, the multiple biometric information images, the multiple pieces of the force distribution information, and the identification information are stored while being associated with one another in the storage circuit 53. Hereinafter, the biometric information images and the force distribution information stored in the storage circuit 53 are referred to as registered images and registered force information. The registered images and the registered force information correspond to each other. The registered force information is the force distribution information detected by the force sensor 20 when the original image of the predetermined part F corresponding to the registered image is captured by the optical sensor 40. A plurality of the registered images, a plurality of pieces of the registered force information, and the identification information are stored for at least one user while being associated with one another in the storage circuit 53.

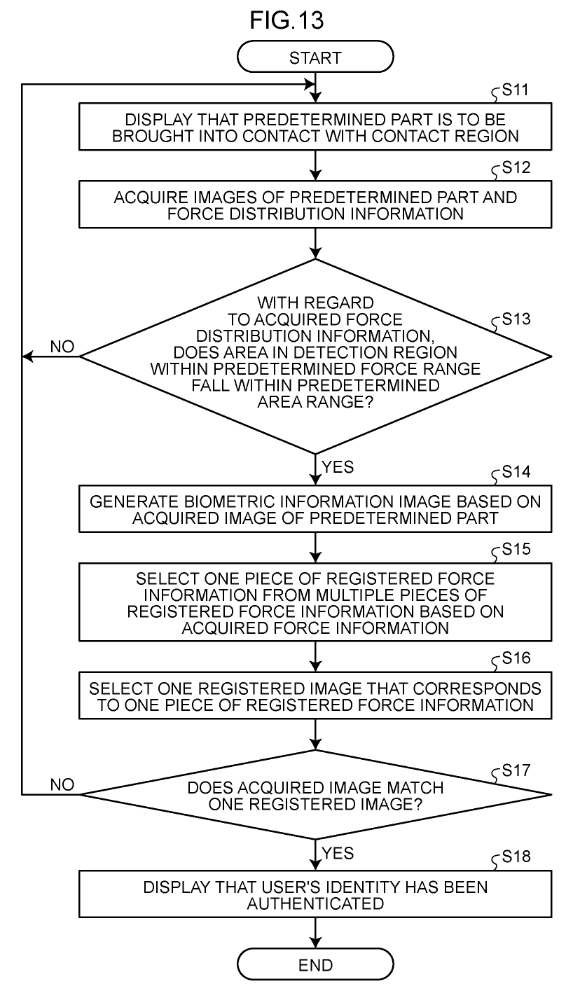

FIG. 13 is a flowchart of a process executed by the control device 50 in the authentication mode. The control device 50 executes the process of the flowchart illustrated in FIG. 13 in response to the selection of the authentication mode. When authenticating a user, the control device 50 performs one-to-one authentication in which the user is authenticated using a registered image and registered force information corresponding to the user. When selecting the authentication mode, the user enters the identification information described above into the input device.

At Step S11, the control device 50 (display circuit 56) displays that the predetermined part F is to be brought into contact with the contact region TR. The user brings the predetermined part F into contact with the contact region TR in accordance with the display content of the display circuit 56.

Then, at Step S12, the control device 50 (acquisition circuit 51) acquires the images of the predetermined part F output by the optical sensor 40 and the force distribution information output by the force sensor 20. The control device 50 acquires the images and the force distribution information of the predetermined part F one by one. The images of the predetermined part F and the force distribution information acquired are synchronized with each other and correspond to each other as described above.

Furthermore, at Step S13, the control device 50 determines, with regard to the acquired force distribution information, whether the area in the detection region DR with force within the predetermined force range falls within the predetermined area range. Specifically, the control device 50 (image processing circuit 52) calculates the area in the detection region DR in which the force falls within the predetermined force range with regard to the force distribution information acquired at Step S12, in the same way as at Step S3 described above.

If the calculated area is outside the predetermined area range (No at Step S13), the control device 50 returns to Step S11. As a result, if the calculated area is outside the predetermined area range, the control device 50 acquires again the image of the predetermined part F output by the optical sensor 40 and the force distribution information output by the force sensor 20 at Step S12. That is, if, with regard to the force distribution information acquired at Step S12, the area in the detection region DR in which the force falls within the predetermined force range is outside the predetermined area range, the determination circuit 54 does not perform the determination to be described later.

In contrast, if the calculated area is within the predetermined area range (Yes at Step S13), the control device 50 proceeds to Step S14. At this time, the control device 50 has acquired the force distribution information in which the calculated area is within the predetermined area range and the image of the predetermined part F corresponding to the force distribution information. If the calculated area is within the predetermined area range, the control device 50 can acquire the image of the predetermined part F in which the blood flow condition of the predetermined part F is appropriate and the vascular pattern can be appropriately recognized in the biometric information image. Therefore, the determination circuit 54 can appropriately perform the determination to be described later.

At Step S14, the control device 50 (image processing circuit 52) generates the biometric information image based on the acquired image of the predetermined part F. Hereinafter, the biometric information image generated at Step S14 is referred to as an "acquired image". In addition, the force distribution information acquired at Step S12 described above is referred to as "acquired force information". The acquired force information is the force distribution information detected by the force sensor 20 when the original image of the predetermined part F corresponding to the acquired image is captured by the optical sensor 40. That is, the acquired image and the acquired force information correspond to each other.

Then, at Step S15, the control device 50 selects one piece of the registered force information from the multiple pieces of the registered force information based on the acquired force information. Specifically, the control device 50 selects one piece of the registered force information that is most similar to the acquired force information from the multiple pieces of the registered force information that correspond to the entered identification information. For example, the control device 50 calculates the range in which the force values of the registered force information overlap the force values of the acquired force information for each of the multiple pieces of the registered force information, and selects the registered force information in which the area of the range is largest in the detection region DR, from among the multiple pieces of the registered force information.

Furthermore, at Step S16, the control device 50 selects one registered image (for example, the biometric information image illustrated in FIG. 11C) that corresponds to one piece of the registered force information.

Then, at Step S17, the control device 50 (determination circuit 54) determines whether the acquired image matches the one registered image. The control device 50 compares the acquired image with the one registered image and determines whether the images match by using a known method. For example, the control device 50 extracts a plurality of feature points of the vascular pattern of the acquired image and a plurality of feature points of the vascular pattern of the one registered image, and determines that the acquired image matches the one registered image if the feature points of the two vascular patterns match at a ratio equal to or greater than a first predetermined ratio. The first predetermined ratio is a ratio at which the vascular pattern of the acquired image can be determined to match the vascular pattern of the one registered image. If the acquired image matches the one registered image, the control device 50 authenticates the user's identity.

If the acquired image is determined to match the one registered image (Yes at Step S17), the control device 50 proceeds to Step S18. At Step S18, the control device 50 (display circuit 56) displays that the user's identity has been authenticated. When the process at Step S18 ends, the control device 50 ends the computer program.

In contrast, at Step S17, if the feature points of both vascular patterns match at a ratio lower than the first predetermined ratio (No at Step S17), the control device 50 determines that the acquired image does not match the one registered image, and returns to Step S11. If a negative determination at Step S16 is made a predetermined number (such as five) of times or more, the control device 50 may end the computer program.

Thus, the control device 50 selects one registered image from the multiple registered images based on the acquired force information and the registered force information, compares the acquired image with the one registered image, and authenticates the user's identity. If the control device 50 does not use the acquired force information and the registered force information, the force of the predetermined part F acting on the contact region TR varies, which may cause relatively large differences between the shape of the vascular pattern in the registered image and the shape of the vascular pattern in the acquired image. For example, if the one registered image corresponds to the biometric information image illustrated in FIG. 11A and the acquired image corresponds to the biometric information image illustrated in FIG. 11C, the ratio at which the feature points of the two images match is smaller than the first predetermined ratio. In this case, the user is not authenticated as himself/herself, even though the user is the person himself/herself.

That is, even if the force of the predetermined part F acting on the contact region TR varies, the control device 50 can appropriately authenticate the user's identity by selecting one registered image from the multiple registered images based on the acquired force information and the registered force information as described above.

As described above, in the registration mode and the authentication mode, the image processing circuit 52 generates the biometric information image including the biometric information of the user based on the image of the predetermined part F captured by the optical sensor 40. In the registration mode, the storage circuit 53 stores therein the biometric information image generated by the image processing circuit 52 and the force detected by the force sensor 20 when the image of the predetermined part F corresponding to the biometric information image is captured, as the registered image and the registered force information corresponding to the registered image. In addition, in the authentication mode, the determination circuit 54 acquires the biometric information image generated by the image processing circuit 52 and the force detected by the force sensor 20 when the image of the predetermined part F corresponding to the biometric information image is captured, as the acquired image and the acquired force information corresponding to the acquired image, and determines whether the acquired image matches the registered image based on the acquired force information and the registered force information.

While the preferred embodiment of the present disclosure has been described above, the present disclosure is not limited to the embodiment described above. The content disclosed in the embodiment is merely an example, and can be variously modified within the scope not departing from the gist of the present disclosure. Any modifications appropriately made within the scope not departing from the gist of the present disclosure also naturally belong to the technical scope of the present disclosure.

For example, the detection system 1 need not include the light source device 30. In this case, external light is reflected in the predetermined part F and enters the optical sensor 40 as the reflected light.

The force detected by the force sensor 20 may be force per unit area generated by contact of the predetermined part F with the contact region TR, or may be a load (pressing force) generated by contact of the predetermined part F with the contact region TR.

The photodiode PD may be a positive-intrinsic-negative (PIN) photodiode.

The control device 50 may be capable of registering the biometric information images and the force distribution information of a plurality of people (N people). In this case, when authenticating a user in the authentication mode, the control device 50 may perform one-to-N authentication using the registered images and the registered force information of the multiple people (N people) stored in the storage circuit 53. That is, if the user is included in the multiple people (N people) registered, the user is identified as the user corresponding to the identification information.

The control device 50 may issue a warning if the calculated area is outside the predetermined area range at Step S13 illustrated in FIG. 12 (No at Step S13). Specifically, the control device 50 (display circuit 56) warns the user by displaying that the pressing force of the predetermined part F against a contact part is not appropriate. The control device 50 may further be provided with a speaker and may warn the user by outputting sound from the speaker.

When the control device 50 selects a plurality of pieces of the force distribution information from the multiple pieces of the acquired force distribution information at Step S3 in FIG. 12, the control device 50 may select, from the multiple pieces of the acquired force distribution information, force distribution information in which the ratio of the range of force values of the force distribution information that fall within a predetermined force range is equal to or greater than a second predetermined ratio. The second predetermined ratio is a range of force values that corresponds to an area in the detection region DR in which the determination circuit 54 can appropriately perform the above-described determination.

At Step S3 in FIG. 12, the control device 50 may select, from the multiple pieces of the acquired force distribution information, force distribution information in which the maximum force value (or median force value) is within the predetermined force range.

At Step S3 in FIG. 12, the control device 50 may select one piece of the force distribution information from the acquired multiple pieces of force distribution information. The control device 50 may select, for example, the force distribution information in which the area in the detection region DR with force within the predetermined force range is largest. In this case, one piece of the force distribution information selected at Step S13 corresponds to the one piece of the registered force information mentioned at Step S15 in FIG. 13.

At Step S13 in FIG. 13, the control device 50 may determine whether the ratio of force values that fall within the predetermined force range to the range of force values in the acquired force distribution information (acquired force information) is equal to or greater than the second predetermined ratio mentioned above. If the ratio of force values that fall within the predetermined force range to the range of force values in the acquired force information is equal to or greater than the second predetermined ratio (Yes at Step S13), the control device 50 proceeds to Step S14. In contrast, if the ratio of force values that fall within the predetermined force range to the range of force values in the acquired force information is smaller than the second predetermined ratio (No at Step S13), the control device 50 returns to Step S11.

At Step S15 in FIG. 13, the control device 50 may calculate, for each of the multiple pieces of the registered force information, a range in which the force values of the registered force information overlap the force values of the acquired force distribution information (acquired force information), and select one piece of the registered force information in which the range is largest. The control device 50 may compare the maximum force values of the multiple pieces of the registered force information with the maximum force value of the acquired force information, and select the registered force information having the maximum force value closest to the maximum force value of the acquired force information. The control device 50 may compare the median force values of the multiple pieces of the registered force information with the median force value of the acquired force information, and select the registered force information having the median force value closest to the median force value of the acquired force information.

At Step S17 in FIG. 13, the control device 50 (image processing circuit 52) may adjust the acquired image and determine whether the adjusted acquired image matches the one registered image. Specifically, the control device 50 first extracts the contour of the predetermined part F in the one registered image selected at Step S16 as described above, based on the registered force information corresponding to the one registered image. The control device 50 then extracts the contour of the predetermined part F in the acquired image as described above based on the acquired force information. Furthermore, the control device 50 adjusts the position and orientation of the predetermined part F in the acquired image so that the position of the contour of the predetermined part F in the acquired image matches the contour of the predetermined part F in the one registered image. This adjustment matches the position and orientation of the vascular pattern in the acquired image with the position and orientation of the vascular pattern in the one registered image. Therefore, even if the position and orientation of the predetermined part F vary relative to the contact region TR, the control device 50 (determination circuit 54) can accurately determine whether the acquired image matches the one registered image.

In the registration mode, the control device 50 may generate, based on two of the multiple pieces of force distribution information selected at Step S3 in FIG. 12, force distribution information having an intermediate force distribution between the two of the multiple pieces of force distribution information. In this case, the control device 50 (image processing circuit 52) generates, based on two biometric information images corresponding to the two pieces of force distribution information, a biometric information image having intermediate biometric information between the two biometric information images. For example, if the control device 50 has acquired the first biometric information image G1 illustrated in FIG. 11A and the third biometric information image G3 illustrated in FIG. 11C and has not acquired the second biometric information image G2 illustrated in FIG. 11B, the control device 50 generates the second biometric information image G2 illustrated in FIG. 11B based on the first biometric information image G1 and the third biometric information image G3. The control device 50 (storage circuit 53) stores the intermediate biometric information image and the intermediate force distribution information in correspondence with each other.

The force sensor 20 may have flexibility to be deformed by the contact of the predetermined part F with the contact region TR. In this case, the array substrate 21, the array electrode 22, and the common electrode 23 have flexibility to be deformed by the contact of the predetermined part F with the contact region TR, so that the force sensor 20 has the flexibility as a whole. In this case, for example, the second substrate 41 may have relatively high rigidity so as to give the optical sensor 40 relatively high rigidity as a whole, or all members constituting the optical sensor 40 may have flexibility to be deformed by the contact of the predetermined part F with the contact region TR so as to give the optical sensor 40 the flexibility as a whole.

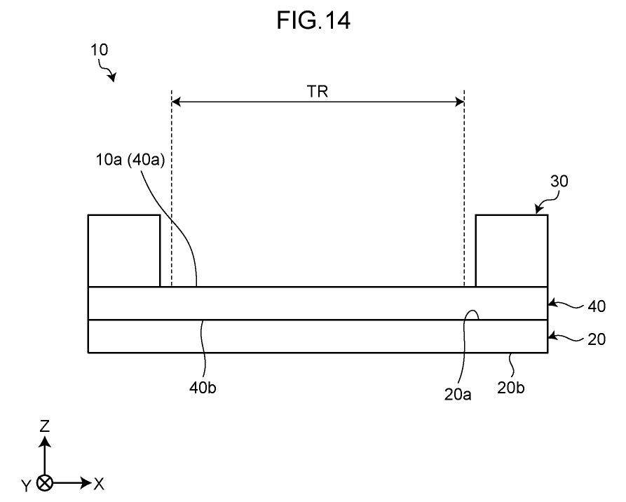

FIG. 14 is a side view of the detection device 10 according to a modification of the embodiment of the present disclosure. In the present modification, the force sensor 20 is positioned on an opposite surface (that is, the back surface 40b) of the optical sensor 40 opposite the imaging surface 40a. In this case, the imaging surface 40a of the optical sensor 40 corresponds to the contact surface 10a of the detection device 10, and the imaging region PR of the optical sensor 40 matches the contact region TR of the detection device 10. In this case, for example, the second substrate 41 may have relatively high rigidity so as to give the optical sensor 40 relatively high rigidity as a whole, or all members constituting the optical sensor 40 may have flexibility to be deformed by the contact of the predetermined part F with the contact region TR so as to give the optical sensor 40 the flexibility as a whole. In this case, the light source device 30 is positioned on the imaging surface 40a of the optical sensor 40. In this case, the force sensor 20 need not have a light-transmitting property. Furthermore, in this case, for example, the first substrate 21a may have relatively high rigidity so as to give the force sensor 20 relatively high rigidity as a whole, or all members constituting the force sensor 20 may have flexibility to be deformed by the contact of the predetermined part F with the contact region TR so as to give the force sensor 20 the flexibility as a whole. Thus, one of the optical sensor 40 and the force sensor 20 may have the flexibility to be deformed by the contact of the predetermined part F with the contact region TR.

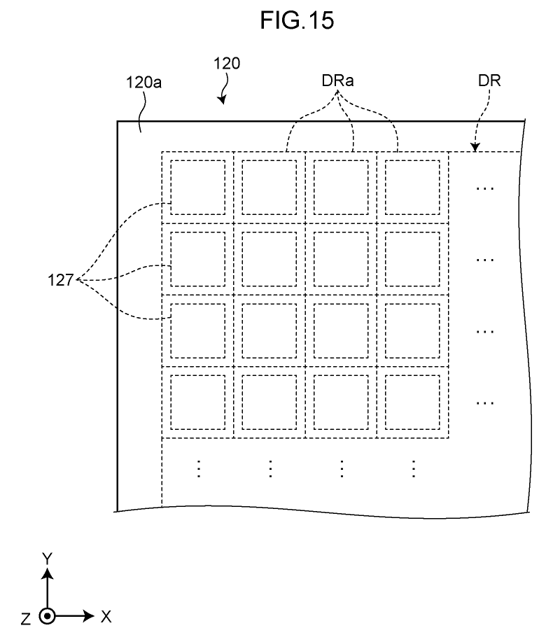

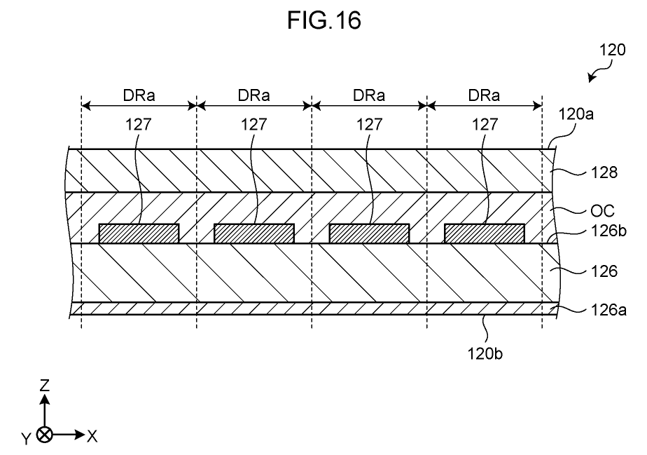

FIG. 15 is a schematic diagram illustrating a configuration of a force sensor 120 according to the modification of the present disclosure. FIG. 16 is a sectional view of the force sensor 120 illustrated in FIG. 15. The force sensor 120 according to the present modification is a capacitive force sensor. The force sensor 120 detects the presence or absence of force for each of the individual detection regions DRa. The force sensor 120 includes a third substrate 126 (corresponding to a "substrate"), a plurality of electrodes 127, and a third protective layer 128.

The third substrate 126 is formed in the same way as the first substrate 21a of the force sensor 20 of the embodiment described above. A shield 126a is positioned on the back surface of the third substrate 126. The shield 126a reduces an influence on capacitance of the electrodes 127 from the back surface side of the force sensor 120. The back surface of the shield 126a corresponds to a back surface 120b of the force sensor 120.

The electrodes 127 are arranged on a front surface 126b (corresponding to a "second surface") of the third substrate 126. One electrode 127 is provided in each of the individual detection regions DRa. That is, the electrodes 127 are arranged in a matrix having a row-column configuration in the detection region DR in plan view. The electrodes 127 are formed of a conductive material such as ITO.

The third protective layer 128 is positioned on the front surface 126b side of the third substrate 126 with an adhesive layer OC interposed therebetween. The third protective layer 128 is formed in the same way as the first protective layer 25 of the force sensor 20 of the embodiment described above. The front surface of the third protective layer 128 corresponds to a detection surface 120a that has the detection region DR.

Each of the electrodes 127 is coupled to the control device 50 via a signal line (not illustrated). If the predetermined part F is not in contact with the detection region DR, the control device 50 detects the electrostatic capacitance of the electrode 127 for each of the individual detection regions DRa. In contrast, if the predetermined part F is in contact with the detection region DR, the electrostatic capacitance increases in the individual detection regions DRa contacted by the predetermined part F. The force sensor 120 detects a change in the electrostatic capacitance generated by the contact of the predetermined part F with the detection region DR. The control device 50 identifies the individual detection region DRa contacted by the predetermined part F based on this change in the electrostatic capacitance.

The greater the force acting on the detection region DR caused by the contact of the predetermined part F with the detection region DR, the greater the area of contact of the predetermined part F in the detection region DR, thereby increasing the number of the individual detection regions DRa contacted by the predetermined part F. That is, the control device 50 can calculate and obtain the magnitude of the force acting on the detection region DR (that is, a force value, equivalent to the pressing force of the predetermined part F per unit area in the detection region DR) based on the number of the individual detection regions DRa contacted by the predetermined part F. That is, the force sensor 120 detects the force acting on the detection region DR, and outputs it as force value information to the control device 50.

In the present modification, at Step S3 in FIG. 12, the control device 50 selects a plurality of pieces of force value information indicating force values within a predetermined force range from among the acquired multiple pieces of the force value information. At Step S6 in FIG. 12, the control device 50 stores the force value information selected at Step S3 as the registered force information.

In the present modification, the control device 50 may determine whether the force value indicated by the acquired force value information (acquired force information) is within the predetermined force range at Step S13 in FIG. 13. If the force value indicated by the acquired force information is within the predetermined force range (Yes at Step S13), the control device 50 proceeds to Step S14. In contrast, if the force value of the acquired force information is outside the predetermined force range (No at Step S13), the control device 50 returns to Step S11.

In the present modification, when the control device 50 selects one piece of the registered force information from the multiple pieces of the registered force information at Step S15 in FIG. 13, the control device 50 selects the registered force information having a force value closest to the force value indicated by the acquired force information.

Other operational advantages accruing from the aspects described in the above-described embodiment that are obvious from the description herein, or that are conceivable as appropriate by those skilled in the art will naturally be understood as accruing from the present disclosure.

What is claimed is:

1. A detection system comprising:
a detection device having a contact region to be contacted by a predetermined part of a user; and
a control device configured to operate in an authentication mode to authenticate the user, wherein
the detection device comprises:
an optical sensor configured to capture an image of the predetermined part that includes a contact zone of the predetermined part in contact with the contact region; and a force sensor configured to detect force generated by contact of the predetermined part with the contact region, and
the control device comprises:
an image processing circuit configured to generate a biometric information image comprising biometric information of the user based on the image of the predetermined part captured by the optical sensor;
a storage circuit configured to store in advance the biometric information image and force information on the force detected by the force sensor when the image of the predetermined part corresponding to the biometric information image is captured, as a registered image and registered force information corresponding to the registered image; and
a determination circuit configured to acquire the biometric information image generated by the image processing circuit and the force information on the force detected by the force sensor when the image of the predetermined part corresponding to the biometric information image is captured, as an acquired image and acquired force information corresponding to the acquired image, and determine whether the acquired image matches the registered image based on the acquired force information and the registered force information, in the authentication mode.

2. The detection system according to claim 1, wherein the control device is further configured to operate, in a registration mode, to store the registered image and the registered force information for at least one person in the storage circuit.

3. The detection system according to claim 1, wherein
the storage circuit is configured to store a plurality of the registered images and a plurality of pieces of the registered force information corresponding to the registered images, and
the determination circuit is configured to select one of the registered images based on the acquired force information and determine whether the acquired image matches the one of the registered images.

4. The detection system according to claim 1, wherein the biometric information is a vascular pattern or a fingerprint pattern.

5. The detection system according to claim 1, wherein the determination circuit is configured not to perform the determination when, with regard to the acquired force information, an area in the contact region in which force falls within a predetermined force range is outside a predetermined area range.

6. The detection system according to claim 1, wherein the control device is configured to issue a warning when, with regard to the acquired force information, an area in the contact region in which force falls within a predetermined force range is outside a predetermined area range.

7. The detection system according to claim 1, wherein the detection device further comprises a light source device configured to emit light toward the predetermined part.

8. The detection system according to claim 1, wherein
the image processing circuit is configured to, in the authentication mode:
extract a contour of the predetermined part in the registered image based on the registered force information;
extract the contour of the predetermined part in the acquired image based on the acquired force information; and
adjust a position and an orientation of the predetermined part in the acquired image so that the position of the contour of the predetermined part in the acquired image matches the contour of the predetermined part in the registered image.

9. The detection system according to claim 1, wherein
the force sensor has a contact surface including the contact region and has a light-transmitting property, and
the optical sensor is positioned on an opposite surface of the force sensor opposite the contact surface.

10. The detection system according to claim 1, wherein one of the optical sensor and the force sensor has flexibility to be deformed by the contact of the predetermined part with the contact region.

11. The detection system according to claim 1, wherein the optical sensor comprises a plurality of photodiodes that overlap the contact region and are arranged in a matrix having a row-column configuration in plan view.

12. The detection system according to claim 11, wherein the photodiodes are organic photodiodes (OPDs).

13. The detection system according to claim 1, wherein
the force sensor comprises:
an array substrate;
a plurality of array electrodes arranged in a matrix having a row-column configuration on a first surface of the array substrate;
a common electrode positioned on the first surface; and
a sensor layer that faces the first surface and is configured to change in electrical resistance value by contact of the predetermined part with the contact region.

14. The detection system according to claim 1, wherein
the force sensor comprises:
a substrate; and
a plurality of electrodes arranged in a matrix having a row-column configuration on a second surface of the substrate, and
the force sensor is configured to detect a change in electrostatic capacitance generated by contact of the predetermined part with the contact region.

15. A detection device comprising
an optical sensor and a force sensor that are configured to be directly or indirectly contacted by a predetermined part of a user, wherein
the optical sensor has an imaging region to capture an image of the predetermined part,
the force sensor has a detection region to detect force generated by being contacted by the predetermined part,
the imaging region overlaps the detection region in plan view, and
timing of outputting the image from the optical sensor is synchronized with timing of outputting the force from the force sensor.

16. The detection device according to claim 15, wherein
the force sensor has a detection surface including the detection region and has a light-transmitting property, and
the optical sensor is positioned on an opposite surface of the force sensor opposite the detection surface.

17. The detection device according to claim 15, wherein one of the optical sensor and the force sensor has flexibility to be deformed by being contacted by the predetermined part.
